(19) United States
(12) Patent Application Publication
(10) Pub. No.: US 2023/0345916 A1
(43) Pub. Date: Nov. 2, 2023

(54) PUPAE DISPENSING SYSTEMS

(71) Applicant: Verily Life Sciences LLC, South San Francisco, CA (US)

(72) Inventors: Peter MASSARO, San Carlos, CA (US); Angus KITCHELL, Golden, CO (US); Johanna OHM, Pacifica, CA (US); Martin LOZANO, Oakland, CA (US); Daniel GREELEY, San Francisco, CA (US); Kevin WALKER, San Jose, CA (US)

(73) Assignee: Verily Life Sciences LLC, South San Francisco, CA (US)

(21) Appl. No.: 18/044,944

(22) PCT Filed: Sep. 14, 2021
(86) PCT No.: PCT/US2021/050270

§ 371 Date: Mar. 10, 2023

Related U.S. Application Data
(60) Provisional application No. 62/706,842, filed on Sep. 14, 2020.

(51) Int. Cl. A01K 29/00; A01K 67/033

(52) U.S. Cl. CPC A01K 29/005; A01K 67/033

(57) ABSTRACT

An insect dispensing system is described. The insect dispensing system includes a plate diverting structure supported by a frame. A first plate of the structure includes a set of channels and a second plate of the structure includes a set of apertures. The plates are aligned such that one end of each channel is aligned with one of the apertures. A population of insect pupae suspended in a liquid is dispensed into the set of channels via an inlet. As the liquid flows through the system, it is distributed among the set of channels, ultimately exiting via the apertures into a set of catch basins.

### CROSS-REFERENCE TO RELATED APPLICATIONS

This application claims priority to U.S. Provisional Patent Application No. 62/706,842, filed Sep. 14, 2020, titled “Pupae Dispensing Device,” the entirety of which is hereby incorporated by reference.

### BACKGROUND

Insects can be reared in a lab environment. For example, as part of sterile insect technique (SIT) program, mosquitos may be reared from larvae to full grown adults. For tracking purposes in rearing factories, it may be desirable to estimate the number of mosquitos during the various stages of development. Because of differences in size, shape, and activity between these stages, one approach to estimating mosquito numbers may not be suitable for all stages.

### BRIEF SUMMARY

Various examples are described relating to devices, systems, and methods for distributing and dispensing a population of insects.

One general aspect includes an insect dispensing system, including a dispensing container that includes a container base including a first surface and a second surface, where a plurality of filter cup holes are formed in the container base and extend between the first surface to the second surface. The dispensing container also includes a perimeter wall connected to the container base and enclosing the first surface to define a dispensing container volume, where at least a portion of the perimeter wall is sloped with respect to the first surface. The insect dispensing system also includes a plurality of filter cups, each including: a filter base including a filtering surface; and a cup perimeter wall connected to the filter base and enclosing the filtering surface to define a filter cup volume, where each filter cup of the plurality of filter cups is removably mountable within one of the plurality of filter cup holes.

Another general aspect includes an insect dispensing container, including: a container base including a first surface and a second surface, where a plurality of filter cup holes is formed in the container base and extend between the first surface to the second surface, where each filter cup hole of the plurality of filter cup holes is configured to removably receive a filter cup. The insect dispensing container also includes a perimeter wall connected to the container base and enclosing the first surface to define a dispensing container volume, where a first portion of the perimeter wall slopes outwardly in a first direction and a second portion of the perimeter wall slopes outwardly in a second direction that is different from the first direction.

Another general aspect includes a method for dispensing insects, including: mounting a plurality of filter cups in a plurality of filter cup holes in a container base of a dispensing container, where the dispensing container includes a perimeter wall that, together with the container base, defines a dispensing container volume. The method also includes positioning the dispensing container within a tub. The method also includes adding an aqueous solution including a population of insect pupae to the dispensing container volume of the dispensing container, where the tub includes a volume of liquid. The method also includes removing, from the dispensing container volume and via the plurality of filter cups, the aqueous solution from the dispensing container volume. The method also includes removing the plurality of filter cups from the plurality of filter cup holes.

Another general aspect includes an insect dispensing system, including: a dispensing container having a container base that includes a plurality of filter cup rails extending longitudinally in the container base, where the plurality of filter cup rails define a plurality of filter cup openings that extend between a first surface to a second surface. The dispensing container also includes a perimeter wall connected to the container base and enclosing the first surface to define a dispensing container volume, where at least a portion of the perimeter wall is sloped with respect to the first surface. The insect dispensing system also includes a plurality of filter cups, each including: a filter base including a filtering surface; and a slide flange connected to the filter base and enclosing the filtering surface to define a filter cup volume, where each filter cup of the plurality of filter cups is slidably and removably mountable within the filter cup opening via a pair of filter cup rails of the plurality of filter cup rails.

Another general aspect includes an insect dispensing container, including: a container base including a first surface and a second surface. The insect dispensing container also includes a plurality of filter cup rails extending longitudinally in the container base, where the plurality of filter cup rails define a plurality of filter cup openings that extend between the first surface to the second surface, where a pair of filter cup rails is configured to removably receive one or more filter cups. The insect dispensing container also includes a perimeter wall connected to the container base and enclosing the first surface to define a dispensing container volume, where a first portion of the perimeter wall slopes outwardly in a first direction and a second portion of the perimeter wall slopes outwardly in a second direction that is different from the first direction.

Another general aspect includes an insect dispensing system, including: a first section including a first inlet, a first outlet, and a second outlet. The insect dispensing system also includes a second section including a second inlet, a third outlet, and a fourth outlet. The insect dispensing system also includes a third section including a third inlet, a fifth outlet, and a sixth outlet. The insect dispensing system also includes a first tube having a first end in fluid communication with the first inlet. The insect dispensing system also includes a second tube having a third end in fluid communication with the first outlet of the first branch and a fourth end in fluid communication with the second inlet of the second branch. The insect dispensing system also includes a third tube having a fifth end in fluid communication with the second outlet of the first branch and a sixth end in fluid communication with the third inlet of the third branch, where each of the third outlet, the fourth outlet, the fifth outlet, and the sixth outlet is positionable to drain liquid introduced at the first inlet into respective catch basins.

Another general aspect includes an insect dispensing system, including: a cylindrical tube including an interior surface and an exterior surface, where a plurality of ports are formed in the cylindrical tube, each port of the plurality of ports extends between the interior surface and the exterior surface. The insect dispensing system also includes a wall connected to the interior surface of the cylindrical tube and extending longitudinally within the cylindrical tube, the wall defining a plurality of peaks and a plurality of valleys, with each valley opening corresponding to a port of the plurality of ports. The insect dispensing system also includes a plurality of divider walls connected to the interior surface of the cylindrical tube, an end of each divider wall of the plurality of divider walls in physical contact with a respective peak of the plurality of peaks, each divider wall extending laterally from the wall. The insect dispensing system also includes a plurality of outlets connected to the exterior surface in fluid communication with the plurality of ports.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1 illustrates a perspective view of an insect dispensing container for use in an insect dispensing system, according to at least one example.

FIG. 2 illustrates a perspective view of a filter cup for use in the insect dispensing container from FIG. 1, according to at least one example.

FIG. 3 illustrates a side sectional view of an insect dispensing system, according to at least one example.

FIG. 4 illustrates a side sectional view of the insect dispensing system of FIG. 3 in a first state of operation, according to at least one example.

FIG. 5 illustrates a side sectional view of the insect dispensing system of FIG. 3 in a second state of operation, according to at least one example.

FIG. 6 illustrates a side sectional view of the insect dispensing system of FIG. 3 in a third state of operation, according to at least one example.

FIG. 7 illustrates a side sectional view of the insect dispensing system of FIG. 3 in a fourth state of operation, according to at least one example.

FIG. 8 illustrates a perspective view of an insect dispensing system including a plurality of filter cups, according to at least one example.

FIG. 9 illustrates a side sectional view of the insect dispensing system of FIG. 8, according to at least one example.

FIG. 10 illustrates a side sectional view of the insect dispensing system of FIG. 8, according to at least one example.

FIG. 11 illustrates a perspective view of an insect dispensing system, according to at least one example.

FIG. 12 illustrates a perspective view of an insect dispensing system, according to at least one example.

FIG. 13 illustrates an end view of the insect dispensing system of FIG. 12, according to at least one example.

FIG. 14 illustrates an end view of the insect dispensing system of FIG. 12, according to at least one example.

FIG. 15 illustrates a flow chart showing a process for dispensing a population of insects, according to at least one example.

FIG. 16 illustrates a generic computing device for interacting with insect dispensing systems, according to at least one example.

FIG. 17 illustrates a side perspective view of an insect dispensing system, according to at least one example.

FIG. 18 illustrates a top perspective view of the insect dispensing system of FIG. 17, according to at least one example.

FIG. 19 illustrates two perspective view of an insect dispensing system, according to at least one example.

### DETAILED DESCRIPTION

Examples are described herein in the context of insect dispensing systems for use in separating and dispensing a population of insects such as mosquito pupae. Those of ordinary skill in the art will realize that the following description is illustrative only and is not intended to be in any way limiting. For example, the techniques described herein can be used to dispense mosquitos in other stages of development and/or other insects. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

In an insect rearing program, the number of insects that may be grouped together without harming the insects at different stages of development may differ. For example, as larvae, mosquitos may be densely packed without harming the larvae (e.g., large quantity of insects per square inch). As pupae, a less dense arrangement having a smaller quantity of insects per cubic inch may be desirable. And as adults, an even less dense arrangement having an even smaller quantity of insects per cubic inch may be desirable. Thus, at each stage is may be desirable to divide the insects into groups consisting of different quantities. The techniques described herein are suitable for dividing a population of pupae that are suspended in a liquid (e.g., water) into a plurality of groups having approximately equal numbers. Depending on the dimensions of a dispensing container, the systems described herein may be scaled to divide any suitable number of pupae (e.g., hundreds, thousands, tens of thousands, or even more) into any suitable number of groups (e.g., two or more).

Turning now to a particular example, an insect dispensing system includes a dispensing container such as a rectangular box. A base of the box includes multiple holes. Each hole is sized and configured to removably receive a filter cup. Each filter cup includes a porous bottom connected to a solid wall. The filter cups are installed from the inside of the box and pushed into the holes until a shoulder on each filter cup contacts the base of the box. The number of filter cups and holes defines the number of groups into which a population of pupae will be divided. The box also includes at least two opposing walls that are joined to the base at oblique angles (e., closer to each other at the bottom of the walls and further apart at the top).

In practice, the box is placed into a larger container (e.g., a tub) that holds a volume of water. Because water can pass through the porous bottoms of the filter cups, when the box is placed in the tub, the inside volume of the box fills with water. The depth of water is selected such that the water does not overtake the tops of the walls of the box. At this point, a population of pupae is added to the interior volume of the box. Naturally, the pupae will gravitate near the surface of the water, leaving a thin film of pupae at the top of the box. The water is then drained or pumped from the tub, which draws the water and the pupae down towards the filter cups at the bottom of the box. Because the walls of the box are sloped towards the base, the film of pupae is constrained (e.g., its surface area is reduced) as it moves toward the filter cups. As the last of the water is drained from the box, the population of pupae is generally equally distributed into the cups.

The insect dispensing system described herein is able to generally equally distribute a population of insects much faster than conventional approaches. For example, one conventional approach requires a human user to count pupae by pupae or a different conventional approach requires an insect singulation and counting system to singulate and count pupae within a stream of water before being diverted to a holding chamber. The insect dispensing system is able to take a large quantity of insect pupae and distribute that quantity into a preset number of groups with little human intervention and in a manner that is faster than a human can or a singulation and counting system can do. In most cases, the accuracy achieved using the insect dispensing system is adequate for purposes of SIT programs.

This illustrative example is given to introduce the reader to the general subject matter discussed herein and the disclosure is not limited to this example. The following sections describe various additional, non-limiting examples of insect dispensing systems.

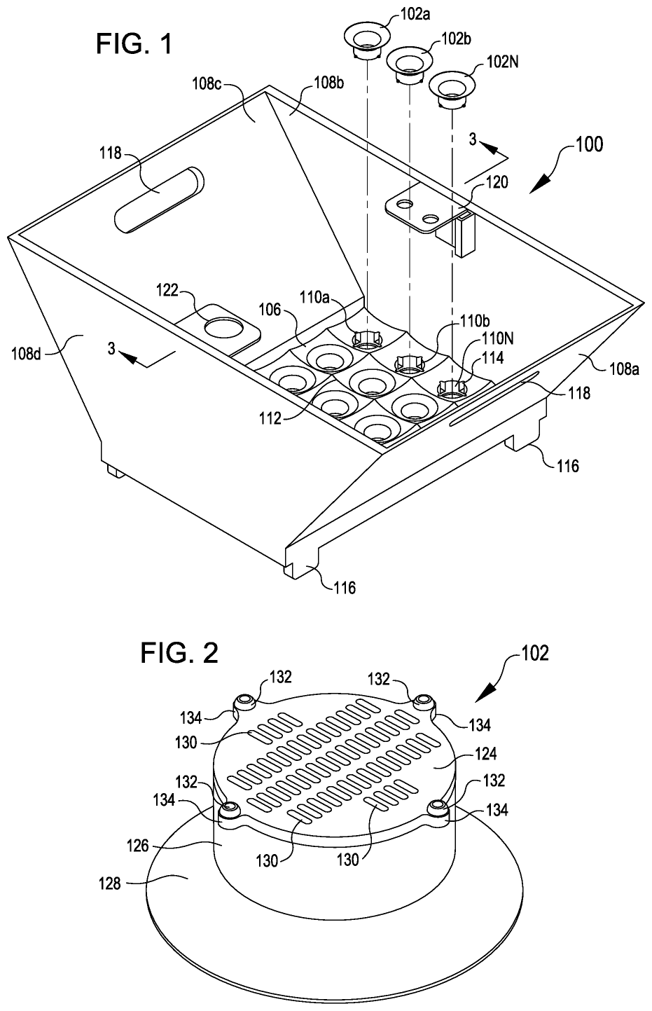

Referring now to the figures, FIG. 1 illustrates a perspective view of an insect dispensing container 100, according to at least one example. The insect dispensing container 100 may be used in an insect dispensing system (e.g., FIG. 3) to divide a population of insects among a plurality of filter cups 102a-102N. The insect dispensing container 100 takes the form of a box with a base 106 and a wall 108. At a top surface of the base 106 is defined a plurality of filter cup holes 110a-110N, a few of which are labeled. The filter cup holes 110 extend through the entirety of the base 106. In FIG. 1, three filter cups 102 have been removed from their corresponding filter cup holes 110. The other filter cup holes 110 in the base 106 are illustrated with their filter cups 102 removably mounted therein. The filter cups 102 may be installed from the top of the base 106 and may be held within the filter cup holes 110 via an interference fit between exterior surfaces of the filter cups 102 and interior surfaces of the filter cup holes 110. Each filter cup hole 110 may also include a mounting structure 114, as labeled with respect to one filter cup hole 110. The mounting structure 114 may include alignment groove(s), a tapered bore with or without a shoulder, a counter-bore with or without a shoulder, and/or any other suitable structure to retain and/or align a filter cup 102 in a filter cup hole 110. Any suitable number of filter cup holes 110 may be arranged in the base 106 in any suitable manner. For example, the filter cup holes 110 may be arranged in an array of columns and rows that is M×N, with M being one or more and N being two or more. In some examples, the distribution of the filter cup holes 110 is uniform to maximize area of the base 106 for filtering.

As illustrated in FIG. 1, the base 106 includes a plurality of funnels 112 formed around each filter cup hole 110. Because of the design of the funnels 112, the top surface of the base 106 has a “waffle” pattern, with raised ridges separating one funnel 112 from another and downward-sloping ramps extending from the raised ridges towards each filter cup hole 110. The funnels 112 are provided to minimize horizontal surfaces on the base 106 and to direct flows of pupae and water towards the filter cup holes 110. In some examples, the top surface of the base 106 is defined as having a plurality of raised ridges surrounding a plurality of wells, with the bottoms of the wells aligned with the filter cup holes 110.

The wall 108 may be formed from a plurality of wall segments 108a-108d. As illustrated, at least two opposing wall segments (e.g., 108a and 108c) may be connected to the base 106 at an oblique angle. In this manner, the wall segments 108a and 108c may slope outward from the base 106. In some examples, the other opposing wall segments 108b and 108d are connected to the base 106 in a similar manner, forming a similar angle with the base 106 or a different angle. The wall segments 108 and the base 106 together define an interior volume of the insect dispensing container 100. Because at least some wall segments 108 are sloped, a cross-sectional area measured at the top of the wall segments 108 (e.g., furthest from the base 106) is greater than a cross-sectional area of the base 106. When water having a population of pupae therein is drained from the insect dispensing container 100, via the filter cups 102 held within the filter cup holes 110, the population of pupae is constrained because of the change in cross-sectional area with respect to elevation. This change naturally forces the density of the pupae to increase, and because of the surface of the base 106 includes the funnels 112, distribute evenly among the plurality of filter cups 102. Once the pupae have been distributed among the filter cups 102, the filter cups 102 can be removed from the filter cup holes 110 for further processing, e.g., placed into a small container for the pupae to transition to adults. Having the wall segments 108 at oblique angles with respect to the base 106 also gives the pupae enough room to breathe prior to being divided.

The insect dispensing container 100 also includes a set of feet 116, a pair of handles 118, a sensor mounting bracket 120, and a flow mounting bracket 122. The set of feet 116, which may include four or more, may be positioned on the bottom of the insect dispensing container 100 to provide a separation between the bottom surface of the base 106 and a tub or other container into which the insect dispensing container 100 will be placed (e.g., see FIG. 3). The pair of handles 118 may be provided for ease of manipulating the insect dispensing container 100. For example, while the description herein focuses on draining water through the filter cups 110 using a separate tub or container, another approach includes adding the pupae to the interior volume of the insect dispensing container 100 and manually (or in an automated manner) lifting the insect dispensing container 100, e.g., using the handles 118, with respect to the water. Thus, the water elevation may be changed with respect to the base 106 or the elevation of the base 106 may be changed with respect to the water elevation. As described with later figures, the sensor mounting bracket 120 is configured for mounting one or more sensors (e.g., depth sensors, optical sensors, cameras) to monitor conditions of the insect dispensing container 100. The flow mounting bracket 122 is used for mounting a tube or other outlet for transferring a liquid, e.g., liquid including pupae, to the insect dispensing container 100.

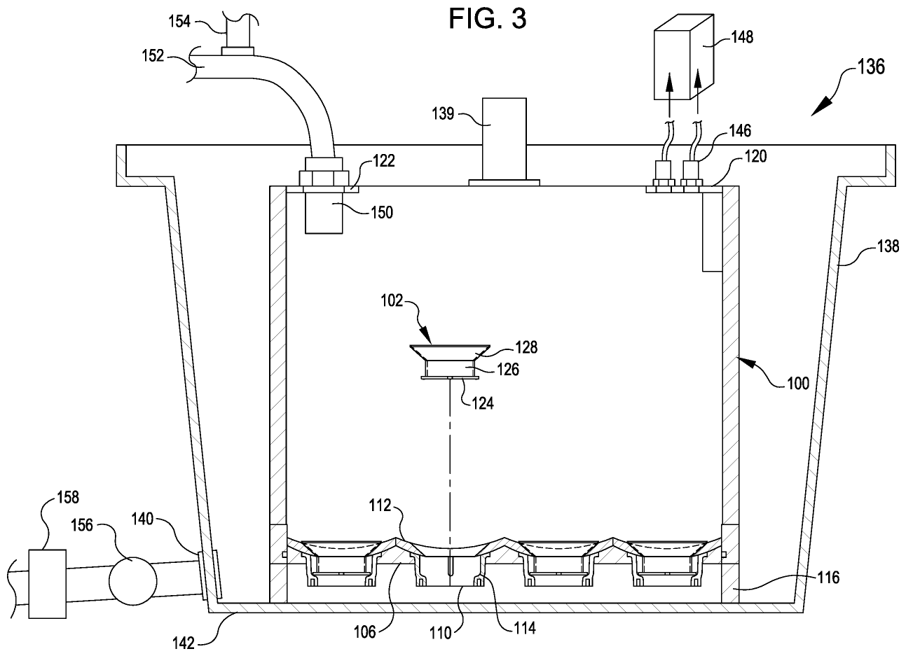

FIG. 2 illustrates a perspective view of a filter cup 102 for use in the insect dispensing container from FIG. 1, according to at least one example. The filter cup 102 in FIG. 2 is illustrated in an upside down orientation, i.e., opposite of the view depicted in FIG. 1. The filter cup 102 includes a base 124 and a perimeter wall 126 that surrounds the base 124 to define an cup interior volume. The perimeter wall 126 is oriented generally in a longitudinal direction. In some examples, the perimeter wall 126 may be fixedly connected to the base 124 or may be removably connected. For example, as illustrated, the base 124 is connected to the perimeter wall 126 via a set of screws 132. The geometry of the base 124, including the bump-outs at which the screws 132 engage with the perimeter wall 126, may be define a plurality of alignment features 134 for aligning a filter cup 102 with a filter cup hole 110. In some examples, the alignment features may be desirable to ensure a correct orientation of the filter cup 102 with respect to the filter cup hole 110.

The filter cup 102 also includes a mounting flange 128 extending laterally and radially outward around the perimeter wall 126. When installed in a filter cup hole 110, the side of the mounting flange 128 visible in FIG. 2 physically contacts a top surface of the base 106 of the insect dispensing container 100. In some examples, this physical contact and, in some cases, contact between the perimeter wall 126 and walls of the filter cup 102 function to retain the filter cup 102 in the filter cup hole 110. Use of the mounting flange 128 in connection with the inwardly sloping ramps surrounding each filter cup hole 110 may create a smooth transition (e.g., a small lip) between the ramps and the filter cup 102. A lower height of the lip may be beneficial to provide unimpeded flow across the lip. In some examples, the mounting flange 128 may slope inwardly towards the perimeter wall 126.

In some examples, in the base 124 is formed a plurality of slots 130 (e.g., pores, openings, or the like) to enable water to pass through the base 124. Each slot 130 may have a lateral width that is greater than an average width of a representative insect larva and less than an average width of a representative insect pupa. In this manner, larvae and other comparably-sized material (e.g., food, waste) will pass through the slots 130, and insect pupae will be prevented from passing through.

The dimensions of the filter cups 102 may be selected based on a desired quantity of pupae to be collected. For example, the filter cups 102 may be configured to hold three thousand to four thousand pupae. In some examples, the number of filter cup holes 110 may depend on the quantity of insects to be divided and the number of groups into which the insects will be divided. For example, the number of filter cup holes 110 may be between 2 and 28, or even greater than 28.

FIG. 3 illustrates a side sectional view of an insect dispensing system 136, according to at least one example. The insect dispensing system 136 includes the insect dispensing container 100, the filter cups 102, and a tub 138, among other components. Generally, the insect dispensing system 136 is used to implement the insect dispensing techniques described herein. The tub 138 may be any suitable container that is large enough to receive the insect dispensing container 100, as shown in FIG. 3. The insect dispensing container 100 may be moveable with respect to the tub 138 via a linear actuator 139.

The tub 138 includes a port 140 located adjacent to a bottom 142 of the tub 138. The insect dispensing container 100 is illustrated with feet 116 resting on the bottom 142. The port 140 may be used to add and/or remove liquid from the tub 138, as described with respect to later figures. For example, the port 140 may be in fluid communication with a valve 156 and/or a pump 158 that can remove liquid from the tub 138 at a consistent rate. In some examples, the valve 156 may be controlled by a solenoid or other automated element to enable controlled opening and closing.

FIG. 3 also depicts the filter cups 102, with three filter cups 102 mounted within corresponding filter cup holes 110 and one filter cup 102 shown in uninstalled state. In this view, the features of the filter cup 102 (e.g., the perimeter wall 126, base 124, and mounting flange 128) and corresponding features of the filter cup hole 110 and the base 106 (e.g., the mounting structure 114 and the funnels 112) are visible. The mounting structure 114 of the filter cup hole 110 may include a counter-bore of sufficient depth corresponding to the height of the perimeter wall 126.

As depicted in FIG. 3, the insect dispensing system 136 also includes one or more sensors 146 mounted to the sensor mounting bracket 120 to monitor conditions of the insect dispensing container 100. The insect dispensing system 136 also includes an inlet port 150 mounted to the flow mounting bracket 122. The inlet port 150 may be configured to deliver a flow of fluid into the insect dispensing container 100. For example, the inlet port 150 may be in fluid communication with a fluid source 152 and an insect source 154. The fluid source 152 may be water or any other suitable fluid. The insect source 154 may include a mechanism such as a funnel for introducing a population of insect pupae into the fluid from the fluid source 152. In some examples, the population of insect pupae are suspended in fluid when introduced from the insect source 154. In some examples, the insect source 154 is the only source connected to the inlet port 150 and is configured to deliver the population of insects to the interior volume of the insect dispensing container 100.

The sensors 146, one or more valves on each of the sources 152 and 154, the valve 156, the linear actuator 139, and/or the pump 158 may be in electrical and/or network communication with a computing device 148. In this manner, the computing device 148 may be configured to control the operation of these elements and manage the flow of fluid and insects into the insect dispensing system 136 and manage flow of fluid out of the insect dispensing system 136. In some examples, the computing device 148 may control the operation of valves and linear actuator 139 based on information received from the sensors.

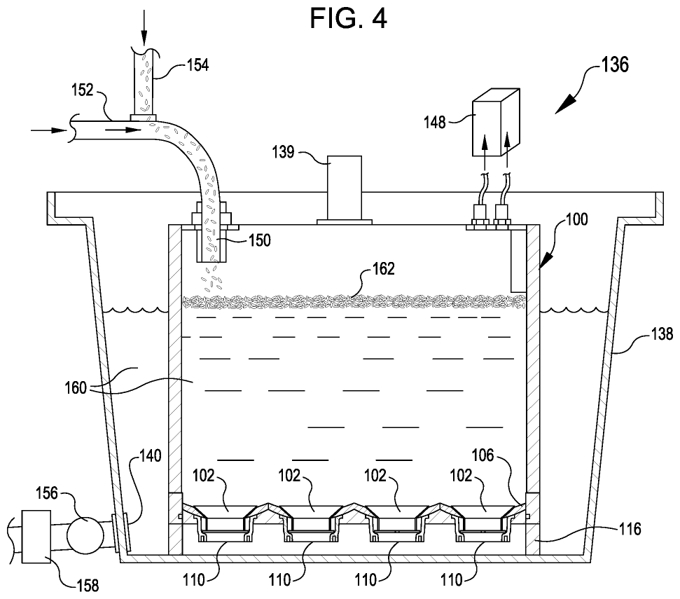

FIGS. 4-7 illustrate a side sectional view of the insect dispensing system of FIG. 3 in various states of operation, according to at least one example. In particular, FIG. 4 illustrates the insect dispensing system at a first state of operation. In the state illustrated in FIG. 4, the valve 156 has been closed and the tub 138 and the insect dispensing container 100 have been filled with a volume of liquid 160. Because of the slots 130 in the bottom of the filter cup holes 102, any liquid introduced into the insect dispensing container 100 will flow through the slots 130 and into the tub 138. Thus, the tub 138 may be filled using the inlet port 150 by liquid from the fluid source 152. In the state illustrated in FIG. 4, an insect population 162 has been added to the interior volume of the insect dispensing container 100. For example, the insect population 162 may have been added via the port 150 from the insect source 154. Given the natural characteristics of the insect population 162, the insects will float at the top of the water level and distribute into a thin film of insects. The film may be multiple insects deep. As described herein, the quantity of insects in the population of insects 162 may depend on a number of factors. In an example use case, the population of insects 162 may be between 80,000 and 110,000 and the number of filter cup holes 110 may be 28 distributed into a four by seven array. At the state illustrated in FIG. 4, the insect dispensing system 136 is ready to begin distributing the insects among the filter cups 102 mounted in the filter cup holes 110.

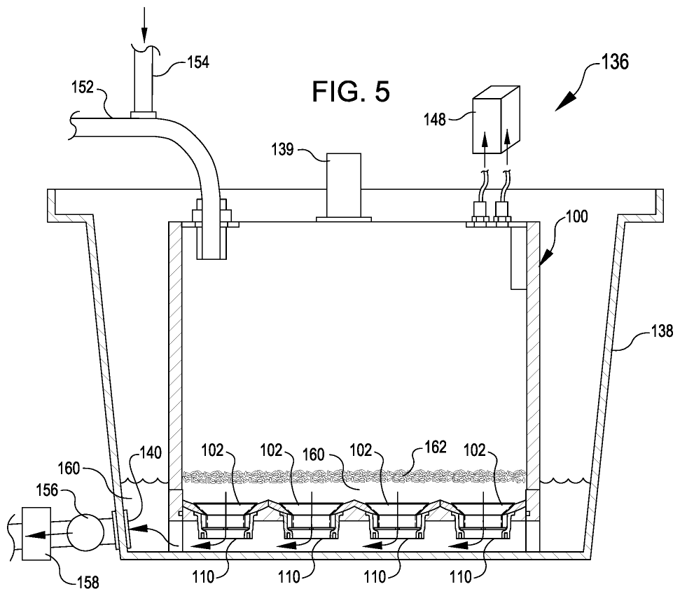

FIG. 5 illustrates the insect dispensing system 136 at a second state of operation subsequent to the first state illustrated in FIG. 4. In the state illustrated in FIG. 5, the valve 156 has been opened and/or the pump 158 has been actuated to begin draining the volume of liquid 160 from the tub 138 and the insect dispensing container 100. For example, if just the valve 156 is used, when the valve 156 is opened, gravity will drain the volume of liquid 160, the rate of which will be dependent on the cross-sectional area of the opening in the port 140. If the pump 158 is included, the rate of drawing out the volume of liquid 160 may be controllable (e.g., faster than the gravity draining or slower than gravity draining).

The volume of liquid 160 is drained via the filter cups 102 in a direction toward the port 140. Because of the sloped wall segments of the insect dispensing container 100, the population of insects 162 is constrained into a smaller and smaller cross-sectional area as the population of insects 162 moves closer to the base 106. This constraining functions to compress the insects sufficiently for distributing the insects among the filter cups 102.

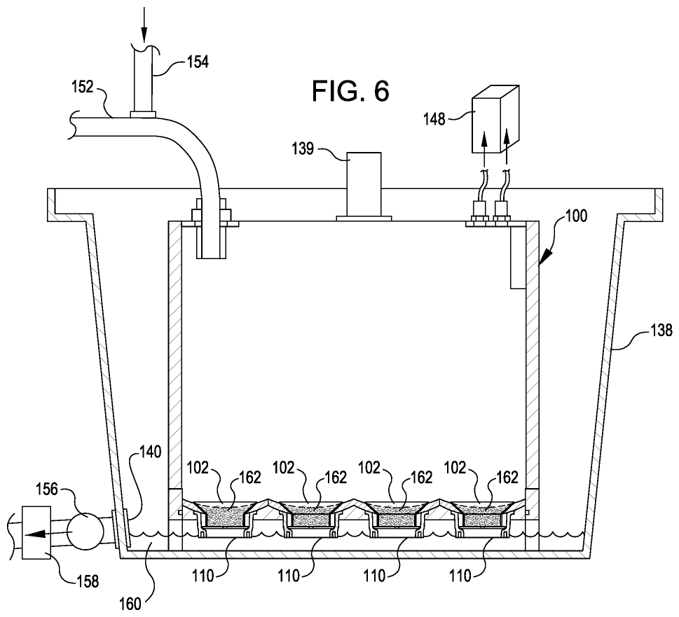

FIG. 6 illustrates the insect dispensing system 136 at a third state of operation subsequent to the second state illustrated in FIG. 5. In the state illustrated in FIG. 6, essentially all of the volume of liquid 160 has been removed from the insect dispensing system 136, which has resulted in the population of insects 162 being distributed among the filter cups 102. Because the distribution of insects in the state illustrated in FIG. 5 was roughly consistent, the distribution of insects among the filter cups 102 is equally roughly consistent.

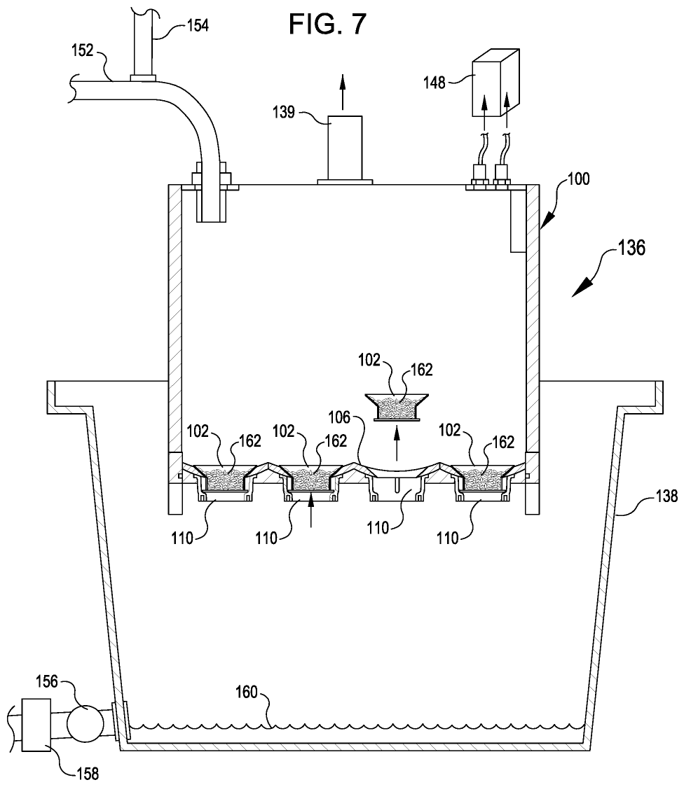

FIG. 7 illustrates the insect dispensing system 136 at a fourth state of operation subsequent to the third state illustrated in FIG. 6. In the state illustrated in FIG. 7, the insect dispensing container 100 has been removed from the tub 138 (e.g., via the linear actuator 139) and one of the filter cups 102 has been removed from its filter cup hole 110. For example, after the insect dispensing container 100 has been lifted out of the tub 138, a removal force may be applied to the base 124 of the filter cup 110 to overcome the forces holding the filter cup 110 in the filter cup hole 110.

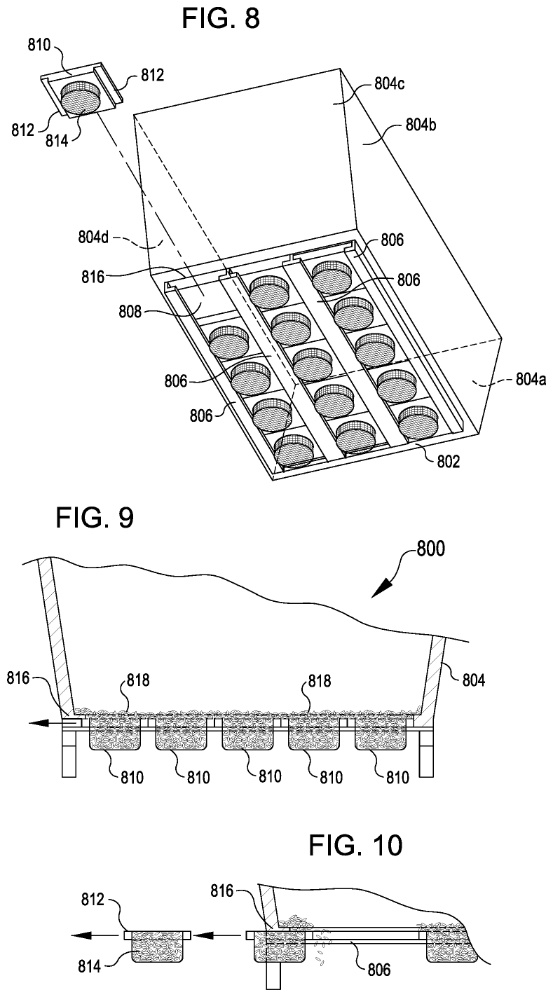

FIGS. 8, 9, and 10 respectively illustrate a perspective view, a side sectional view, and a zoomed-in side sectional view of an insect dispensing container 800 including a plurality of filter cups 810, according to at least one example. In some examples, the insect dispensing container 800 may include only a single filter cup 810, rather than more than one as depicted in FIGS. 8, 9, and 10. The insect dispensing container 800 may have a shape and design that is similar to the insect dispensing container 100 (e.g., a base 802 and a plurality of wall segments 804a-804d connected to the base 802, with at least one of the wall segments 804 connected to the base 802 at an oblique angle). The base 802 includes a plurality of filter cup rails 806 extending longitudinally within the base 802. The filter cup rails 806 define filter cup openings 808, e.g., the space between longitudinal filter cup rails 806. The filter cup rails 806 are configured to slidably receive filter cups 810 into the filter cup openings 808. For example, as illustrated in FIG. 8, three filter cup openings, defined by four filter cup rails 806 are used to hold fifteen filter cups 810 (e.g., five filter cups 810 per filter cup opening 808). Each filter cup 810 includes a slide flange 812 and a filter base 814. The filter base 814 may be include filter openings (e.g., slots) like the filter cups 102. The slide flange 812 includes an angle plate, channel, or groove that slidably engages with a corresponding plate, channel, groove, or other structure of the slide flange 812.

As depicted in FIG. 8, the filter cups 810 are slidably and removably mountable by sliding via an end of the base 802. The insect dispensing container 800 may be used in connection with the insect dispensing system 136 (e.g., in place of the insect dispensing container 100). As depicted in FIG. 9, the filter cups 810 may be filled in a similar manner as described with reference to earlier figures. Once the filter cups 810 are full of insects 818, the filter cups 810 may be removed from the filter cup rails 806 via a sliding motion. A filter cup rail 816 of the base is positioned laterally across the base 802 to scrape off the top most layer of the insects 818 to ensure a uniform quantity of insects by and between the filter cups 810.

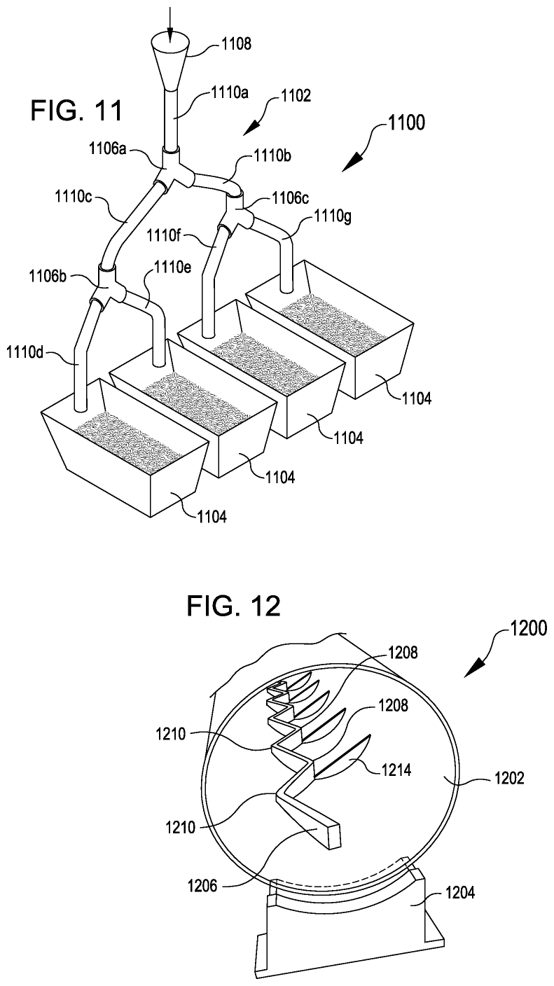

FIG. 11 illustrates a perspective view of an insect dispensing system 1100, according to at least one example. Generally, the insect dispensing system 1100 is configured to divide a population of insects equally among a plurality of catch basins 1104. This system operates on the principle that an equal distribution of insects may be equally distributed into the basins 1104 by going through a dividing structure 1102 including a plurality of Y-shaped dividers 1106.

A population of insect pupae is introduced to the dividing structure 1102 via a funnel 1108. The pupae flow from the funnel 1108 via a tube section 1110a towards a first Y-shaped divider 1106a. When the pupae encounter the first Y-shaped divider 1106a, about half of the population continues towards the Y-shaped divider 1106b via the second tube section 1110c and the other half continues towards the Y-shaped divider 1106c via a third tube section 1110b. These “halves” of the pupae population are halved again at the Y-shaped dividers 1106b and 1106c and flow via the tube sections 1110d-1110g into respective basins 1104. While illustrated in a generally vertical orientation, the dividing structure 1102 may be oriented in a generally horizontal orientation, with a slight slope running from the funnel 1108 to the distal ends of the tube sections 1110d-1110g. In the generally horizontal orientation, the population of insect pupae may be divided with better precision considering that the flow is slower and has fewer turbulent air bubbles.

In some examples, the ratio of inlets to outlets in the insect dispensing system 1100 may be 1-3, 1-7, or 1-n where ‘n’ is the number of outlets from a split, rather than the 1-2 shown in FIG. 11. For example, rather than Y-outlets, the insect dispensing system 1100 may include a plate that includes an inlet and multiple outlets connected with fluid paths. Each outlet may drain into its own basin 1104 located below the respective outlet. In this manner, a single inlet may be used to immediately divide a population of insects between any suitable number of basins.

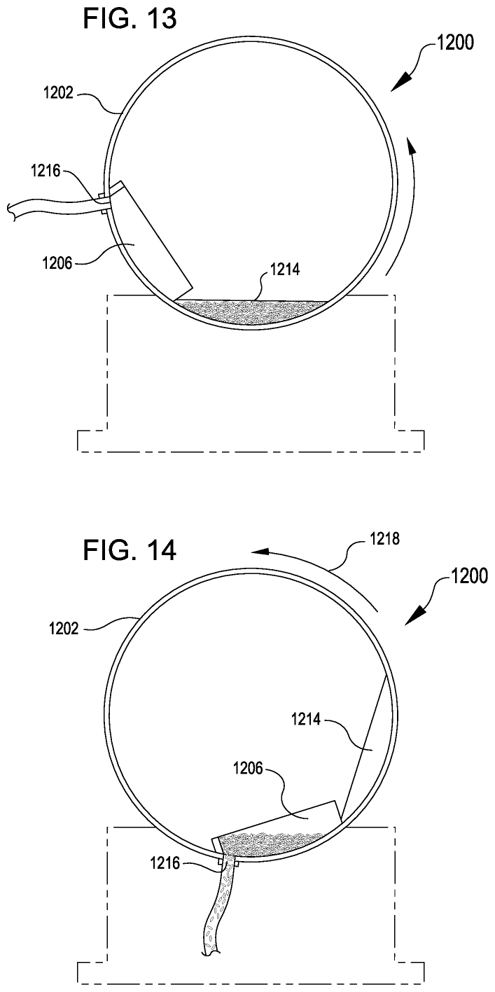

FIGS. 12, 13, and 14 respectively illustrate a perspective view, a first end view, and a second end view of an insect dispensing system 1200, according to at least one example. The insect dispensing system 1200 includes a cylindrical tube 1202 supported by a support structure 1204. Within the interior of the cylindrical tube 1202 is mounted a diverter wall 1206 that extends longitudinally within the cylindrical tube 1202. The diverter wall 1206 includes a plurality of peaks 1208 and a plurality of valleys 1210. The system 1200 also includes divider walls 1214 (e.g., partial circles) extending from each peak 1208 at an oblique angle. As illustrated in FIG. 13, at each valley 1210 is located an outlet 1216 extending through the cylindrical tube 1202. The outlets 1216 may be configured to drain pupae and liquid that accumulate in a volume defined by the diverter wall 1206 and one or more of the divider walls 1214. In operation, the pupae and liquid are added to the cylindrical tube 1202 via one end of the tube 1202. After which, the tube 1202 may be rotated in the direction indicated by arrow 1218. This rotation may cause the pupae to accumulate in the volume defined above. As illustrated in FIG. 14, the pupae may be released from each of these volumes via the outlet 1216, which may include a valve.

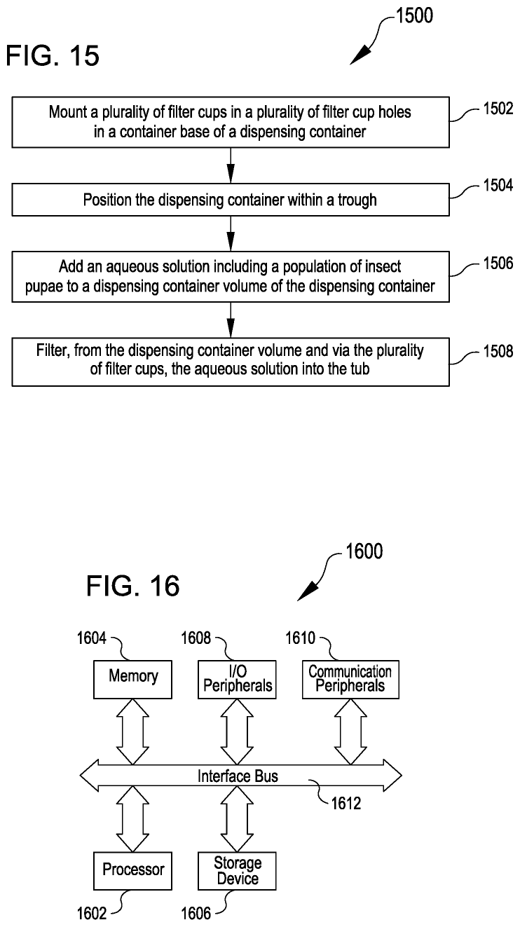

FIG. 15 illustrates a flow chart showing a process 1500 for dispensing a population of insects using an insect dispensing system, according to at least one example. The process 1500 begins at block 1502 by mounting a plurality of filter cups in a plurality of filter cup holes in a container base of a dispensing container. In some examples, the dispensing container includes a perimeter wall that together with the container base defines a dispensing container volume.

At block 1504, the process 1500 includes positioning the dispensing container within a tub. The tub may be sized and configured to receive the dispensing container. In some examples, the tub may have a volume of liquid such as water held therein.

At block 1506, the process 1500 includes adding an aqueous solution including a population of insect pupae to the dispensing container volume of the dispensing container. In some examples, the tub includes a volume of liquid.

At block 1508, the process 1500 includes filtering, from the dispensing container volume and via the plurality of filter cups, the aqueous solution into the tub. In some examples, filtering the aqueous solution may include removing from the dispensing container volume distributes the population of insect pupae among the plurality of filter cups. In some examples, filtering the aqueous solution may include translating the dispensing container from a first position in which the container base is in a volume of liquid in the tub to a second position in which the container base is out of the volume of liquid. In some examples, translating the dispensing container from the first position to the second position includes instructing a linear actuator connected to the dispensing container to move the dispensing container.

In some examples, the process 1500 may further include adding a first portion of the volume of liquid to the tub and a second portion of the volume of liquid to the dispensing container volume. In some examples, the volume of liquid is added via the same port through which the population of insect pupae is added. In some examples, removing the aqueous solution and the first portion of the volume of liquid from the dispensing container volume may include actuating a pump in fluid communication with the volume of liquid to pump the aqueous solution and the first and second portions of the volume of liquid.

At block 1510, the process 1500 includes removing the plurality of filter cups from the plurality of filter cup holes. In some examples, each filter cup of the plurality of filter cups may include a filtering surface connected to a cup perimeter wall that encircles the filtering surface to define a filter cup volume. In some examples, mounting the plurality of filter cups in the plurality of filter cup holes comprises applying an insertion force at the cup perimeter walls that inserts the plurality of filter cups into the plurality of filter cup holes at least until the filtering surfaces are positioned below the container base and the cup perimeter walls physically contact interior surfaces of the plurality of filter cup holes. In some examples, removing the plurality of filter cups from the plurality of filter cup holes may include applying a removal force at the filtering surfaces to translate the plurality of filter cups within the plurality of filter cup holes at least until the cup perimeter walls are removed from the plurality of filter cup holes.

FIG. 16 illustrates examples of components of a computer system 1600, according to at least one example. The computer system 1600 may be a single computer such as a user computing device and/or can represent a distributed computing system such as one or more server computing devices. The computer system 1600 is an example of the computing device 148.

The computer system 1600 may include at least a processor 1602, a memory 1604, a storage device 1606, input/output peripherals (I/O) 1608, communication peripherals 1610, and an interface bus 1612. The interface bus 1612 is configured to communicate, transmit, and transfer data, controls, and commands among the various components of the computer system 1600. The memory 1604 and the storage device 1606 include computer-readable storage media, such as Random Access Memory (RAM), read-only memory (ROM), electrically erasable programmable read-only memory (EEPROM), hard drives, CD-ROMs, optical storage devices, magnetic storage devices, electronic non-volatile computer storage, for example Flash® memory, and other tangible storage media. Any of such computer-readable storage media can be configured to store instructions or program codes embodying aspects of the disclosure. The memory 1604 and the storage device 1606 also include computer-readable signal media. A computer-readable signal medium includes a propagated data signal with computer-readable program code embodied therein. Such a propagated signal takes any of a variety of forms including, but not limited to, electromagnetic, optical, or any combination thereof. A computer-readable signal medium includes any computer-readable medium that is not a computer-readable storage medium and that can communicate, propagate, or transport a program for use in connection with the computer system 1600.

Further, the memory 1604 includes an operating system, programs, and applications. The processor 1602 is configured to execute the stored instructions and includes, for example, a logical processing unit, a microprocessor, a digital signal processor, and other processors. The memory 1604 and/or the processor 1602 can be virtualized and can be hosted within another computing system of, for example, a cloud network or a data center. The I/O peripherals 1608 include user interfaces, such as a keyboard, screen (e.g., a touch screen), microphone, speaker, other input/output devices, and computing components, such as graphical processing units, serial ports, parallel ports, universal serial buses, and other input/output peripherals. The I/O peripherals 1608 are connected to the processor 1602 through any of the ports coupled to the interface bus 1612. The communication peripherals 1610 are configured to facilitate communication between the computer system 1600 and other computing devices over a communications network and include, for example, a network interface controller, modem, wireless and wired interface cards, antenna, and other communication peripherals.

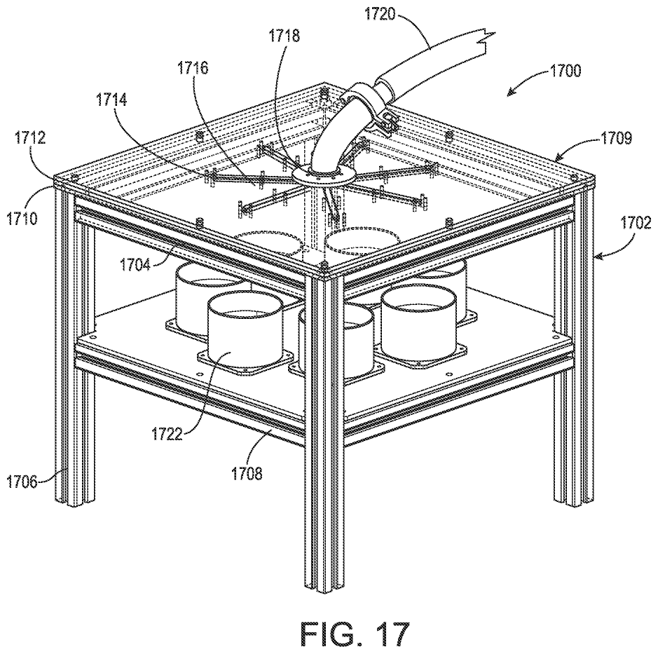

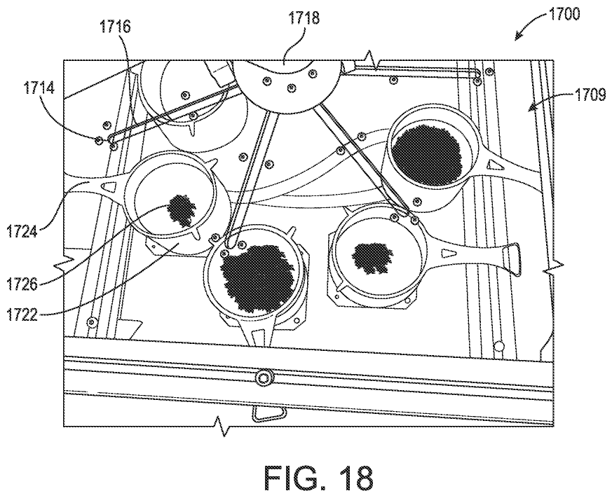

FIG. 17 illustrates a side perspective view of an insect dispensing system 1700, according to at least one example. FIG. 18 illustrates a top perspective view of the insect dispensing system 1700, according to at least one example. The insect dispensing system 1700 may generally be configured to divide a population of insect pupae generally equally into a plurality of groups. Thus, like the other insect dispensing systems described herein, the insect dispensing system 1700 can be used as part of a SIT program or otherwise when any suitable type of insects needs to be divided into groups.

The insect dispensing system 1700 includes a frame 1702. The frame 1702 includes a top 1704 supported by a set of legs 1706. The frame 1702 also includes a shelf 1708 that is supported by the set of legs 1706. The frame 1702, at tops of the legs 1706 and/or along a top surface of the top 1704, support a plate diverting structure 1709. The plate diverting structure 1709 is formed from a bottom plate 1710 and a top plate 1712. The two plates 1710, 1712 may be generally planar and, in some examples, may be connected together using one or more fasteners. In some examples, the two plates 1710, 1712 may be parallel. In some examples, a third plate may be provided that sandwiches the top plate 1712 with the bottom plate 1710. The third plate may form a lid on the channels described herein that are formed in the top plate 1712.

The plate diverting structure 1709 is configured to direct liquid flow between a port inlet 1718 and a set of catch basins 1722 (shown supported by the shelf 1708). To this end, the top plate 1712 may include a set channels 1716, slits, or the like that have an elongated profile and extend outward from the port inlet 1718. The channels 1718 may extend radially with respect to the port inlet 1718. In some examples, other distributions of the channels 1718 that are non-radial may be used. As noted above, in some examples, the channels 1718 may extend through the full width of the top plate 1712. In some examples, only a portion of the channels 1718 may extend through the full width of the top plate 1712. The bottom plate 1710 may include a set of ports or apertures 1714 extending through the full width of the bottom plate 1710. The individual apertures 1714 may be positioned adjacent distal ends of the channels 1716 (e.g., the ends furthest from the inlet port 1718). This positioning may put individual apertures 1714 in fluid communication with the individual channels 1716. The port 1718 is in fluid communication with proximal ends of the channels 1716. In this manner, fluid that enters via the port 1718 may be diverted equally among the set of channels 1716 and flow distally through the channels 1716 until reaching the apertures 1714, at which point the fluid will exit the plate diverting structure 1709 and fall into the catch basins 1722. For example, when the fluid includes a population of insect pupae such as mosquitoes, the flow of the fluid through the plate-diverting structure 1709 may cause the insect pupae to naturally divide into any suitable number of catch basins 1722, depending on the number of channels 1716 and apertures 1714.

The port 1718 may be in fluid communication with a fluid source (such as the fluid source 152 and an insect source (such as the insect source 154) at a location adjacent to or upstream from connecting pipe 1720. The fluid source may be water or any other suitable fluid. The insect source may include a mechanism such as a funnel for introducing a population of insect pupae into the fluid from the fluid source. In some examples, the population of insect pupae are suspended in fluid when introduced from the insect source. In some examples, the insect source is the only source connected to the port 1718 and is configured to deliver the population of insects to the interior volume of the insect dispensing container 100.

As shown in FIG. 18, each catch basin 1722 may be configured to support a strainer 1724 sized and configured to strain liquid such as water and retain insect pupae such as mosquito pupae 1726. The plate diverting structure 1709 may be used to equally distribute the pupae 1726 into the strainers 1724 supported by the catch basin 1722.

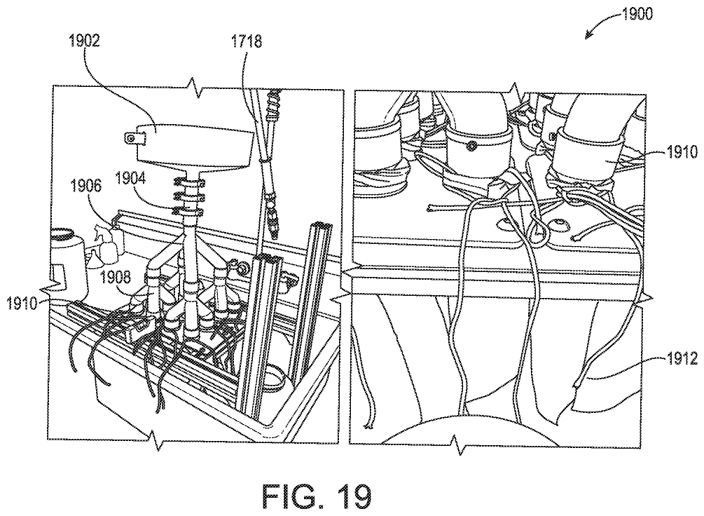

FIG. 19 illustrates two perspective views of an insect dispensing system 1900, according to at least one example. The insect dispensing system 1900 may function similar to the insect dispensing system 1100. Thus, the insect dispensing system 1900 includes a funnel 1902, a tube section 1904, a divider 1906, a set of dividers 1908, and a set of outlets 1910. At a bottom side of each outlet 1910, a catch basin 1912 may be attached (or disposed below with an air gap). The catch basins 1912 may be formed from a pliable material and, in some examples, may be bags formed from mesh material. The mesh may be sized to strain the liquid while keeping the insect pupae. In some examples, the catch basins 1912 are formed from a rigid material, having a fixed shape.

In operation, a population of insect pupae suspended in a liquid such as water may be introduced into the insect dispensing system 1900 at the funnel 1902. Gravity draws the liquid from the funnel 1902 into the tube section 1904 and towards the divider 1906. At the divider 1906, the liquid is divided into the number of outlets provided by the divider 1906. Thus, in this example because the divider 1906 includes four outlets, the liquid will be divided into four parts. The liquid then continues through the outlets of the divider 1906 and toward the set of dividers 1908. At each of the dividers 1908, the liquid is again separated into four outlets before being strained through the catch basins 1912. The number of outlets at each divider 1908 and 1906 and the number of dividers 1908 and 1906 are tunable parameters. Thus, in some examples, the first divider 1906 may include more than four outlets or fewer than four outlets. Similarly, the second dividers 1908 may include more than four outlets or fewer than four outlets. This insect dispensing system 1900 illustrated may divide a population of pupae sixteen ways.

The specification and drawings are, accordingly, to be regarded in an illustrative rather than a restrictive sense. It will, however, be evident that various modifications and changes may be made without departing from the broader spirit and scope of the disclosure as set forth in the claims.

In the following, further examples are described to facilitate the understanding of the present disclosure.

Example 1. In this example, there is provided an insect dispensing system, comprising:
a dispensing container comprising:
   a container base comprising a first surface and a second surface, wherein a plurality of filter cup holes are formed in the container base and extend between the first surface to the second surface; and
   a perimeter wall connected to the container base and enclosing the first surface to define a dispensing container volume, wherein at least a portion of the perimeter wall is sloped with respect to the first surface; and
a plurality of filter cups, each comprising:
   a filter base comprising a filtering surface; and
   a cup perimeter wall connected to the filter base and enclosing the filtering surface to define a filter cup volume, wherein each filter cup of the plurality of filter cup is removably mountable within one of the plurality of filter cup holes.

Example 2. In this example, there is provided an insect dispensing system of any of the preceding or subsequent examples, wherein the plurality of filter cup holes is arranged into M×N array, with M being greater than or equal to 1 and N being greater than or equal to 2.

Example 3. In this example, there is provided an insect dispensing system of any of the preceding or subsequent examples, wherein an area surrounding at least one filter cup hole of the plurality of filter cup holes comprises a raised ridge and a plurality of sloped ramps extending between the raised ridge and a top elevation of the at least one filter cup hole.

Example 4. In this example, there is provided an insect dispensing system of any of the preceding or subsequent examples, wherein the first surface of the container base comprises a repeating three-dimensional waffle pattern comprising raised ridges and recessed wells.

Example 5. In this example, there is provided an insect dispensing system of any of the preceding or subsequent examples, wherein each filter cup hole of the plurality of filter cup holes is formed in the container base with a top elevation at a low point within the recessed wells.

Example 6. In this example, there is provided an insect dispensing system of any of the preceding or subsequent examples, wherein each filter cup hole narrows with respect to depth from the first surface to the second surface.

Example 7. In this example, there is provided an insect dispensing system of any of the preceding or subsequent examples, further comprising a tub sized to receive the dispensing container and configured to hold a volume of liquid.

Example 8. In this example, there is provided an insect dispensing system of any of the preceding or subsequent examples, wherein the tub comprises an outlet, and wherein the system further comprises a pump in fluid communication with the outlet and configured to remove the volume of liquid from the tub.

Example 9. In this example, there is provided an insect dispensing system of any of the preceding or subsequent examples, wherein removing the volume of liquid from the tub causes a population of pupae present in a portion of the volume of liquid within the dispensing container volume to distribute among the plurality of filter cups.

Example 10. In this example, there is provided an insect dispensing system of any of the preceding or subsequent examples, further comprising an actuator connected to the dispensing container and configured to translate the container base vertically with respect to a base of the tub.

Example 11. In this example, there is provided an insect dispensing system of any of the preceding or subsequent examples, further comprising one or more sensors to measure a level of a liquid within the dispensing container volume with respect to the container base.

Example 12. In this example, there is provided an insect dispensing system of any of the preceding or subsequent examples, wherein the portion of the perimeter wall comprises:
a first portion that slopes outwardly from the first surface in a first direction with respect to the first surface; and
a second portion that slopes outwardly from the first surface in a second direction that is different from the first direction.

Example 13. In this example, there is provided an insect dispensing system of any of the preceding or subsequent examples, wherein a plurality of openings is formed in the filtering surface.

Example 14. In this example, there is provided an insect dispensing system of any of the preceding or subsequent examples, wherein the plurality of openings comprises a plurality of slots each having a lateral width that is greater than an average width of a representative insect larva and less than an average width of a representative insect pupa.

Example 15. In this example, there is provided an insect dispensing system of any of the preceding or subsequent examples, wherein the filter cup volume for each filter cup of the plurality of filter cups is substantially the same.

Example 16. In this example, there is provided an insect dispensing system of any of the preceding or subsequent examples, wherein each filter cup is removably mountable within one of the plurality of filter cup holes via an interference fit.

Example 17. In this example, there is provided an insect dispensing system of any of the preceding or subsequent examples, wherein the cup perimeter wall extends longitudinally away from the filter base, and wherein each filter cup further comprises a mounting flange connected to the cup perimeter wall and extending laterally outward around the cup perimeter wall.

Example 18. In this example, there is provided an insect dispensing system of any of the preceding or subsequent examples, wherein bottom sides of the mounting flanges physically contact the first surface of the container base when the plurality of filter cups are mounted within the plurality of filter cup holes.

Example 19. In this example, there is provided an insect dispensing system of any of the preceding or subsequent examples, wherein a counter-bore is formed in the base coaxially with each of the plurality of filter cups holes, and top sides of the mounting flanges are substantially equal in elevation with a portion of the first surface adjacent to the counter-bore when the plurality of filter cups are mounted within the plurality of filter cup holes.

Example 20. In this example, there is provided an insect dispensing system an insect dispensing container, comprising:
a container base comprising a first surface and a second surface, wherein a plurality of filter cup holes is formed in the container base and extend between the first surface to the second surface, wherein each filter cup hole of the plurality of filter cup holes is configured to removably receive a filter cup; and
a perimeter wall connected to the container base and enclosing the first surface to define a dispensing container volume, wherein:
a first portion of the perimeter wall slopes outwardly from the container base in a first direction; and
a second portion of the perimeter wall slopes outwardly from the container base in a second direction that is different from the first direction.

Example 21. In this example, there is provided an insect dispensing system of any of the preceding or subsequent examples, wherein the plurality of filter cup holes is arranged a two-dimensional array defined by M×N, with M being greater than or equal to 1 and N being greater than or equal to 2.

Example 22. In this example, there is provided an insect dispensing system of any of the preceding or subsequent examples, wherein an area surrounding at least one filter cup hole of the plurality of filter cup holes comprises a raised ridge and a plurality of sloped ramps extending between the raised ridge and a top elevation of the at least one filter cup hole.

Example 23. In this example, there is provided an insect dispensing system of any of the preceding or subsequent examples, wherein the first surface of the container base comprises a repeating three-dimensional waffle pattern comprising raised ridges and recessed wells.

Example 24. In this example, there is provided an insect dispensing system of any of the preceding or subsequent examples, wherein each filter cup hole of the plurality of filter cup holes is formed in the container base with a top elevation at a low point within the recessed wells.

Example 25. In this example, there is provided an insect dispensing system of any of the preceding or subsequent examples, wherein each filter cup hole defines a taper from the first surface to the second surface.

Example 26. In this example, there is provided an insect dispensing system of any of the preceding or subsequent examples, wherein a first area enclosed by a top edge of the perimeter wall is greater than a second area of the container base measured at the first surface.

Example 27. In this example, there is provided a method for dispensing insects, comprising:
mounting a plurality of filter cups in a plurality of filter cup holes in a container base of a dispensing container, wherein the dispensing container comprises a perimeter wall that together with the container base defines a dispensing container volume;
positioning the dispensing container within a tub;
adding an aqueous solution comprising a population of insect pupae to the dispensing container volume of the dispensing container; and
filtering, from the dispensing container volume and via the plurality of filter cups, the aqueous solution into the tub.

Example 28. In this example, there is provided a method for dispensing insects of any of the preceding or subsequent examples, wherein filtering the aqueous solution into the tub comprises removing the aqueous solution from the tub, and wherein filtering the aqueous solution distributes the population of insect pupae among the plurality of filter cups.

Example 29. In this example, there is provided a method for dispensing insects of any of the preceding or subsequent examples, wherein filtering the aqueous solution into the tub comprises translating the dispensing container from a first position in which the container base is in a volume of liquid in the tub to a second position in which the container base is out of the volume of liquid.

Example 30. In this example, there is provided a method for dispensing insects of any of the preceding or subsequent examples, wherein translating the dispensing container from the first position to the second position comprises causing a linear actuator connected to the dispensing container to move the dispensing container.

Example 31. In this example, there is provided a method for dispensing insects of any of the preceding or subsequent examples, further comprising adding a first portion of a volume of liquid to the tub and a second portion of the volume of liquid to the dispensing container volume.

Example 32. In this example, there is provided a method for dispensing insects of any of the preceding or subsequent examples, wherein filtering the aqueous solution into the tub comprises actuating a pump in fluid communication with the first portion of the volume of liquid to pump the aqueous solution and the first and second portions of the volume of liquid from the tub.

Example 33. In this example, there is provided a method for dispensing insects of any of the preceding or subsequent examples, wherein each filter cup of the plurality of filter cups comprises a filtering surface connected to a cup perimeter wall that encircles the filtering surface to define a filter cup volume.

Example 34. In this example, there is provided a method for dispensing insects of any of the preceding or subsequent examples, wherein mounting the plurality of filter cups in the plurality of filter cup holes comprises applying an insertion force at the cup perimeter walls that inserts the plurality of filter cups into the plurality of filter cup holes at least until the filtering surfaces are positioned below the container base and the cup perimeter walls physically contact interior surfaces of the plurality of filter cup holes.

Example 35. In this example, there is provided a method for dispensing insects of any of the preceding or subsequent examples, wherein removing the plurality of filter cups from the plurality of filter cup holes comprises applying a removal force at the filtering surfaces to translate the plurality of filter cups within the plurality of filter cup holes at least until the cup perimeter walls are removed from the plurality of filter cup holes.

Example 36. In this example, there is provided an insect dispensing system, comprising:
a dispensing container having a container base and comprising:
a plurality of filter cup rails extending longitudinally in the container base, wherein the plurality of filter cup rails define a plurality of filter cup openings that extend between a first surface to a second surface; and
a perimeter wall connected to the container base and enclosing the first surface to define a dispensing container volume, wherein at least a portion of the perimeter wall is sloped with respect to the first surface; and
a plurality of filter cups, each comprising:
a filter base comprising a filtering surface; and
a slide flange connected to the filter base and enclosing the filtering surface to define a filter cup volume, wherein each filter cup of the plurality of filter cup is slidably and removably mountable within the filter cup opening via a pair of filter cup rails of the plurality of filter cup rails.

Example 37. In this example, there is provided an insect dispensing system of any of the preceding or subsequent examples, further comprising a wiper ledge disposed adjacent to a portion of the perimeter wall and extending laterally with respect to the plurality of filter cup rails, the wiper ledge configured to remove insects that extend above the filter cup volume.

Example 38. In this example, there is provided an insect dispensing container, comprising:
a container base comprising a first surface and a second surface;
a plurality of filter cup rails extending longitudinally in the container base, wherein the plurality of filter cup rails define at least one filter cup opening that extends between the first surface to the second surface, wherein a pair of filter cup rails is configured to removably receive at least one filter cup; and
a perimeter wall connected to the container base and enclosing the first surface to define a dispensing container volume, wherein:
   a first portion of the perimeter wall slopes outwardly from the container base in a first direction; and
   a second portion of the perimeter wall slopes outwardly from the container base in a second direction that is different from the first direction.

Example 39. In this example, there is provided an insect dispensing system comprising:
a first section comprising a first inlet, a first outlet, and a second outlet;
a second section comprising a second inlet, a third outlet, and a fourth outlet;
a third section comprising a third inlet, a fifth outlet, and a sixth outlet;
a first tube having a first end in fluid communication with the first inlet and a second end opposite the first end;
a second tube having a third end in fluid communication with the first outlet of the first branch and a fourth end in fluid communication with the second inlet of the second branch; and
a third tube having a fifth end in fluid communication with the second outlet of the first branch and a sixth end in fluid communication with the third inlet of the third branch, wherein each of the third outlet, the fourth outlet, the fifth outlet, and the sixth outlet is positionable to drain liquid introduced at the first inlet into respective catch basins.

Example 40. In this example, there is provided an insect dispensing system comprising:
a cylindrical tube comprising an interior surface and an exterior surface, wherein a plurality of ports are formed in the cylindrical tube, each port of the plurality of ports extends between the interior surface and the exterior surface;
a wall connected to the interior surface of the cylindrical tube and extending longitudinally within the cylindrical tube, the wall defining a plurality of peaks and a plurality of valleys, with each valley opening corresponding to a port of the plurality of ports;
a plurality of divider walls connected to the interior surface of the cylindrical tube, an end of each divider wall of the plurality of divider walls in physical contact with a respective peak of the plurality of peaks, each divider wall extending laterally from the wall; and
a plurality of outlets connected to the exterior surface in fluid communication with the plurality of ports.

Example 41. In this example, there is provided an insect dispensing system comprising:
a first section comprising a first inlet, a first outlet, and a second outlet;
a second section comprising a second inlet, a third outlet, and a fourth outlet;
a third section comprising a third inlet, a fifth outlet, and a sixth outlet;
a first tube having a first end in fluid communication with the first inlet and a second end opposite the first end;
a second tube having a third end in fluid communication with the first outlet of the first branch and a fourth end in fluid communication with the second inlet of the second branch; and
a third tube having a fifth end in fluid communication with the second outlet of the first branch and a sixth end in fluid communication with the third inlet of the third branch, wherein each of the third outlet, the fourth outlet, the fifth outlet, and the sixth outlet is positionable to drain liquid introduced at the first inlet into respective catch basins.

Example 42. In this example, there is provided an insect dispensing system of any of the preceding or subsequent examples, wherein the respective catch basins comprise strainer bags attached to each of the third outlet, the fourth outlet, the fifth outlet, and the sixth outlet.

Example 43. In this example, there is provided an insect dispensing system of any of the preceding or subsequent examples, wherein the first inlet comprises a funnel.

Example 44. In this example, there is provided an insect dispensing system of any of the preceding or subsequent examples, further comprising a fluid source and an insect source, wherein the first inlet is in fluid communication with the fluid source and the insect source.

Example 45. In this example, there is provided an insect dispensing system of any of the preceding or subsequent examples, wherein the fluid source comprises a mechanism to introduce fluid into the first inlet, and wherein the insect source comprises a mechanism to introduce a population of insect pupae into the fluid.

Example 46. In this example, there is provided an insect dispensing system of any of the preceding or subsequent examples, wherein the plurality of individual streams of fluid distribute the population of insect pupae among the plurality of catch basins when the population of insect pupae and the fluid flow from the inlet port towards the plurality of apertures

Example 47. In this example, there is provided an insect dispensing system, comprising:
a cylindrical tube comprising an interior surface and an exterior surface, wherein a plurality of ports are formed in the cylindrical tube, each port of the plurality of ports extends between the interior surface and the exterior surface;
a wall connected to the interior surface of the cylindrical tube and extending longitudinally within the cylindrical tube, the wall defining a plurality of peaks and a plurality of valleys, with each valley opening corresponding to a port of the plurality of ports;
a plurality of divider walls connected to the interior surface of the cylindrical tube, an end of each divider wall of the plurality of divider walls in physical contact with a respective peak of the plurality of peaks, each divider wall extending laterally from the wall; and
a plurality of outlets connected to the exterior surface in fluid communication with the plurality of ports.

Example 48. In this example, there is provided an insect dispensing system, comprising:
a frame;
an inlet port;
a first plate supported by the frame and comprising a plurality of apertures extending through the first plate;
a second plate supported by the first plate and comprising a plurality of elongated channels formed therein, wherein first ends of the elongated channels intersect with the inlet port and second ends of the elongated channels intersect with respective apertures of the plurality of apertures; and
a plurality of catch basins positioned below respective apertures of the plurality of apertures to capture a plurality of individual streams of fluid that flow from the inlet port via respective elongated channels of the plurality of elongated channels and out the plurality of apertures.

Example 49. In this example, there is provided an insect dispensing system of any of the preceding or subsequent examples, wherein the second plate is connected to the first plate.

Example 50. In this example, there is provided an insect dispensing system of any of the preceding or subsequent examples, wherein the plurality of elongated channels are open channels or closed channels.

Example 51. In this example, there is provided an insect dispensing system of any of the preceding or subsequent examples, wherein the inlet port is configured to direct liquid into the plurality of elongated channels adjacent to the first ends.

Example 52. In this example, there is provided an insect dispensing system of any of the preceding or subsequent examples, wherein the plurality of apertures and the plurality of elongated channels together define a plurality of liquid flow channels, with a surface of the first plate forming a bottom surface of the plurality of liquid flow channels.

Example 53. In this example, there is provided an insect dispensing system of any of the preceding or subsequent examples, further comprising a shelf supported by the frame, and wherein the plurality of catch basins are supported by the shelf.

Example 54. In this example, there is provided an insect dispensing system of any of the preceding or subsequent examples, further comprising a fluid source and an insect source, wherein the inlet port is in fluid communication with the fluid source and the insect source.

Example 55. In this example, there is provided an insect dispensing system of any of the preceding or subsequent examples, wherein the fluid source comprises a mechanism to introduce fluid into the inlet port, and wherein the insect source comprises a mechanism to introduce a population of insect pupae into the fluid.

Example 56. In this example, there is provided an insect dispensing system of any of the preceding or subsequent examples, wherein the plurality of individual streams of fluid distribute the population of insect pupae among the plurality of catch basins when the population of insect pupae and the fluid flow from the inlet port towards the plurality of apertures.

Example 57. In this example, there is provided an insect dispensing system of any of the preceding or subsequent examples, further comprising a plurality of filter cups removably mounted within the catch basins, wherein the plurality of filter cups are configured to filter materials suspended in the plurality of individual streams.

Example 58. In this example, there is provided an insect dispensing system of any of the preceding or subsequent examples, wherein each of the catch basins comprises an outlet for draining liquid from the respective catch basin.

Example 59. In this example, there is provided an insect dispensing system of any of the preceding or subsequent examples, wherein a number of the plurality of apertures is equal to a number of the plurality of elongated channels.

Example 60. In this example, there is provided an insect dispensing system of any of the preceding or subsequent examples, further comprising a third plate supported by the second plate such that the second plate is disposed between the third plate and the first plate.

Example 61. In this example, there is provided an insect dispensing system of any of the preceding or subsequent examples, wherein an aperture is formed in the third plate, and the inlet port is mounted to the third plate.

Example 62. In this example, there is provided an insect dispensing system of any of the preceding or subsequent examples, wherein the plurality of apertures, the plurality of elongated channels, and the third plate together define a plurality of liquid flow channels, with a first surface of the first plate forming a bottom surface of the plurality of liquid flow channels and a second surface of the third plate forming a top surface of the plurality of liquid flow channels.

Other variations are within the spirit of the present disclosure. Thus, while the disclosed techniques are susceptible to various modifications and alternative constructions, certain illustrated examples thereof are shown in the drawings and have been described above in detail. It should be understood, however, that there is no intention to limit the disclosure to the specific form or forms disclosed, but on the contrary, the intention is to cover all modifications, alternative constructions, and equivalents falling within the spirit and scope of the disclosure, as defined in the appended claims.

The use of the terms “a” and “an” and “the” and similar referents in the context of describing the disclosed examples (especially in the context of the following claims) are to be construed to cover both the singular and the plural, unless otherwise indicated herein or clearly contradicted by context. The terms “comprising,” “having,” “including,” and “containing” are to be construed as open-ended terms (e.g., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate examples of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is otherwise understood within the context as used in general to present that an item, term, etc., may be either X, Y, or Z, or any combination thereof (e.g., X, Y, and/or Z). Thus, such disjunctive language is not generally intended to, and should not, imply that certain examples require at least one of X, at least one of Y, or at least one of Z to each be present.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and all three of A and B and C.

Preferred examples of this disclosure are described herein, including the best mode known to the inventors for carrying out the disclosure. Variations of those preferred examples may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. An insect dispensing system, comprising:
a frame;
an inlet port;
a first plate supported by the frame and comprising a plurality of apertures extending through the first plate;
a second plate supported by the first plate and comprising a plurality of elongated channels formed therein, wherein first ends of the elongated channels intersect with the inlet port and second ends of the elongated channels intersect with respective apertures of the plurality of apertures; and
a plurality of catch basins positioned below respective apertures of the plurality of apertures to capture a plurality of individual streams of fluid that flow from the inlet port via respective elongated channels of the plurality of elongated channels and out the plurality of apertures.

2. The insect dispensing system of claim 1, wherein the second plate is connected to the first plate.

3. The insect dispensing system of claim 1, wherein the plurality of elongated channels are open channels or closed channels.

4. The insect dispensing system of claim 1, wherein the inlet port is configured to direct liquid into the plurality of elongated channels adjacent to the first ends.

5. The insect dispensing system of claim 1, wherein the plurality of apertures and the plurality of elongated channels together define a plurality of liquid flow channels, with a surface of the first plate forming a bottom surface of the plurality of liquid flow channels.

6. The insect dispensing system of claim 1, further comprising a shelf supported by the frame, and wherein the plurality of catch basins are supported by the shelf.

7. The insect dispensing system of claim 1, further comprising a fluid source and an insect source, wherein the inlet port is in fluid communication with the fluid source and the insect source.

8. The insect dispensing system of claim 7, wherein the fluid source comprises a mechanism to introduce fluid into the inlet port, and wherein the insect source comprises a mechanism to introduce a population of insect pupae into the fluid.

9. The insect dispensing system of claim 8, wherein the plurality of individual streams of fluid distribute the population of insect pupae among the plurality of catch basins when the population of insect pupae and the fluid flow from the inlet port towards the plurality of apertures.

10. The insect dispensing system of claim 1, further comprising a plurality of filter cups removably mounted within the catch basins, wherein the plurality of filter cups are configured to filter materials suspended in the plurality of individual streams.

11. The insect dispensing system of claim 1, wherein each of the catch basins comprises an outlet for draining liquid from the respective catch basin.

12. The insect dispensing system of claim 1, wherein a number of the plurality of apertures is equal to a number of the plurality of elongated channels.

13. The insect dispensing system of claim 1, further comprising a third plate supported by the second plate such that the second plate is disposed between the third plate and the first plate.

14. The insect dispensing system of claim 13, wherein an aperture is formed in the third plate, and the inlet port is mounted to the third plate.

15. The insect dispensing system of claim 13, wherein the plurality of apertures, the plurality of elongated channels, and the third plate together define a plurality of liquid flow channels, with a first surface of the first plate forming a bottom surface of the plurality of liquid flow channels and a second surface of the third plate forming a top surface of the plurality of liquid flow channels.

16. An insect dispensing system, comprising:
a first section comprising a first inlet, a first outlet, and a second outlet;
a second section comprising a second inlet, a third outlet, and a fourth outlet;
a third section comprising a third inlet, a fifth outlet, and a sixth outlet;
a first tube having a first end in fluid communication with the first inlet and a second end opposite the first end;
a second tube having a third end in fluid communication with the first outlet of the first branch and a fourth end in fluid communication with the second inlet of the second branch; and
a third tube having a fifth end in fluid communication with the second outlet of the first branch and a sixth end in fluid communication with the third inlet of the third branch, wherein each of the third outlet, the fourth outlet, the fifth outlet, and the sixth outlet is positionable to drain liquid introduced at the first inlet into respective catch basins.

17. The insect dispensing system of claim 16, wherein the respective catch basins comprise strainer bags attached to each of the third outlet, the fourth outlet, the fifth outlet, and the sixth outlet.

18. The insect dispensing system of claim 16, wherein the first inlet comprises a funnel.

19. The insect dispensing system of claim 16, further comprising a fluid source and an insect source, wherein the first inlet is in fluid communication with the fluid source and the insect source.

20. The insect dispensing system of claim 19, wherein the fluid source comprises a mechanism to introduce fluid into the first inlet, and wherein the insect source comprises a mechanism to introduce a population of insect pupae into the fluid.

21. The insect dispensing system of claim 20, wherein the plurality of individual streams of fluid distribute the population of insect pupae among the plurality of catch basins when the population of insect pupae and the fluid flow from the inlet port towards the plurality of apertures

22. An insect dispensing system, comprising:
a dispensing container comprising:
a container base comprising a first surface and a second surface, wherein a plurality of filter cup holes are formed in the container base and extend between the first surface to the second surface; and
a perimeter wall connected to the container base and enclosing the first surface to define a dispensing container volume, wherein at least a portion of the perimeter wall is sloped with respect to the first surface; and
a plurality of filter cups, each comprising:
a filter base comprising a filtering surface; and
a cup perimeter wall connected to the filter base and enclosing the filtering surface to define a filter cup volume, wherein each filter cup of the plurality of filter cup is removably mountable within one of the plurality of filter cup holes.

23. The insect dispensing system of claim 22, wherein an area surrounding at least one filter cup hole of the plurality of filter cup holes comprises a raised ridge and a plurality of sloped ramps extending between the raised ridge and a top elevation of the at least one filter cup hole.

24. The insect dispensing system of claim 22, wherein the first surface of the container base comprises a repeating three-dimensional waffle pattern comprising raised ridges and recessed wells, wherein each filter cup hole of the plurality of filter cup holes is formed in the container base with a top elevation at a low point within the recessed wells.

25. The insect dispensing system of claim 22, further comprising a tub sized to receive the dispensing container and configured to hold a volume of liquid, wherein the tub comprises an outlet, and wherein the system further comprises a pump in fluid communication with the outlet and configured to remove the volume of liquid from the tub, and wherein removing the volume of liquid from the tub causes a population of pupae present in a portion of the volume of liquid within the dispensing container volume to distribute among the plurality of filter cups.

26. The insect dispensing system of claim 22, further comprising an actuator connected to the dispensing container and configured to translate the container base vertically with respect to a base of the tub.

27. The insect dispensing system of claim 22, wherein the portion of the perimeter wall comprises:
a first portion that slopes outwardly from the first surface in a first direction with respect to the first surface; and
a second portion that slopes outwardly from the first surface in a second direction that is different from the first direction.

28. The insect dispensing system of claim 22, wherein a plurality of openings is formed in the filtering surface, and wherein the plurality of openings comprises a plurality of slots each having a lateral width that is greater than an average width of a representative insect larva and less than an average width of a representative insect pupa.

29. A method for dispensing insects, comprising:
mounting a plurality of filter cups in a plurality of filter cup holes in a container base of a dispensing container, wherein the dispensing container comprises a perimeter wall that together with the container base defines a dispensing container volume;
positioning the dispensing container within a tub;
adding an aqueous solution comprising a population of insect pupae to the dispensing container volume of the dispensing container; and
filtering, from the dispensing container volume and via the plurality of filter cups, the aqueous solution into the tub.

30. The method for dispensing insects of claim 29, wherein filtering the aqueous solution into the tub comprises removing the aqueous solution from the tub, and wherein filtering the aqueous solution distributes the population of insect pupae among the plurality of filter cups.

31. The method for dispensing insects of claim 29, wherein filtering the aqueous solution into the tub comprises translating the dispensing container from a first position in which the container base is in a volume of liquid in the tub to a second position in which the container base is out of the volume of liquid.

32. The method for dispensing insects of claim 31, wherein translating the dispensing container from the first position to the second position comprises causing a linear actuator connected to the dispensing container to move the dispensing container.

33. The method for dispensing insects of claim 29, further comprising adding a first portion of a volume of liquid to the tub and a second portion of the volume of liquid to the dispensing container volume.

34. The method for dispensing insects of claim 33, wherein filtering the aqueous solution into the tub comprises actuating a pump in fluid communication with the first portion of the volume of liquid to pump the aqueous solution and the first and second portions of the volume of liquid from the tub.

35. The method for dispensing insects of claim 29, wherein each filter cup of the plurality of filter cups comprises a filtering surface connected to a cup perimeter wall that encircles the filtering surface to define a filter cup volume.
